(19) United States
(12) Patent Application Publication
(10) Pub. No.: US 2026/0060742 A1
(43) Pub. Date: Mar. 5, 2026

(54) BIFURCATED JAW FOR ELECTROSURGICAL INSTRUMENT

(71) Applicant: Cilag GmbH International, Zug (CH)

(72) Inventor: Eric W. Brunner, Cincinnati, OH (US)

(21) Appl. No.: 18/815,908

(22) Filed: Aug. 27, 2024

(51) Int. Cl. A61B 18/14; A61B 18/08

(52) U.S. Cl. CPC A61B 18/1445; A61B 18/085

(57) ABSTRACT

An instrument includes a body assembly, a shaft assembly, and an end effector. The end effector includes a first jaw and a second jaw. The end effector is configured to grasp tissue between the first jaw and the second jaw. The shaft is positioned between the body assembly and the end effector. The second jaw includes a second jaw body, which includes at least one recess. The second jaw further includes an electrode assembly. The electrode assembly includes at least one protrusion extending toward the second jaw body. The at least one protrusion is sized to fit inside a respective recess of the at least one recess and is configured to align the electrode assembly to the second jaw body.

### BACKGROUND

A variety of surgical instruments include a tissue cutting element and one or more elements that transmit radio frequency (RF) energy to tissue (e.g., to coagulate or seal the tissue). Examples of such electrosurgical instruments and related concepts are disclosed in U.S. Pat. No. 6,500,176 entitled “Electrosurgical Systems and Techniques for Sealing Tissue,” issued Dec. 31, 2002, the disclosure of which is incorporated by reference herein, in its entirety; U.S. Pat. No. 9,526,565, entitled “Electrosurgical Devices,” issued Dec. 27, 2016, the disclosure of which is incorporated by reference herein, in its entirety; U.S. Pat. No. 11,857,247, entitled “Jaw for Surgical Instrument End Effector,” issued Jan. 2, 2024, the disclosure of which is incorporated by reference herein, in its entirety; and U.S. Pub. No. 2022/0008120, entitled “Electrosurgical Instrument with Floating Jaw Component,” published Jan. 13, 2022, the disclosure of which is incorporated by reference herein in its entirety.

An electrosurgical instrument may be powered by an external generator. Examples of such generators are disclosed in U.S. Pat. No. 8,986,302, entitled “Surgical Generator for Ultrasonic and Electrosurgical Devices,” issued Mar. 24, 2015, the disclosure of which is incorporated by reference herein, in its entirety.

Some electrosurgical instruments may be supported and driven by a robotic surgical system. Examples of robotically controlled electrosurgical instruments are disclosed in U.S. Pat. No. 11,576,738, entitled “Systems and Instruments for Tissue Sealing,” issued Feb. 14, 2023, the disclosure of which is incorporated by reference herein in its entirety; and U.S. Pub. No. 2022/0338891, entitled “Systems for Setting Jaw Gap in Surgical Tool End Effectors,” published Oct. 27, 2022, the disclosure of which is incorporated by reference herein in its entirety.

Alignment of components within electrosurgical instruments may be important to promote appropriate function and longevity of the instrument. Controlling alignment of components to achieve these results may be achieved at a design phase by incorporating fitments between components such that they are self-aligning with one another.

Molds that are used in injection molding processes may include a gate that allows the molding material to enter the mold cavity. The molding material may harden within the gate and remain integral with the primary molded part after the molding process is complete. In some cases, the hardened molding material from the gate may be removed from the primary molded part, as this piece of material may otherwise present an appendage that serves no useful purpose. It may therefore be desirable to provide some useful purpose to a hardened molding material from an injection mold gate, such that the hardened molding material from the injection mold gate is not removed from the primary molded part.

While a variety of surgical instruments have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements.

FIG. 1 depicts a schematic view of an example of an electrosurgical instrument

FIG. 2A depicts a perspective view of an end effector and distal portion of a shaft assembly of the instrument of FIG. 1, with the end effector in an open configuration.

FIG. 2B depicts a perspective view of the end effector and distal portion of the shaft assembly of FIG. 2A, with the end effector in a closed configuration.

FIG. 2C depicts a perspective view of the end effector and distal portion of the shaft assembly of FIG. 2A, with the end effector in the closed configuration and a knife in a distal position.

FIG. 3 depicts a perspective view of a first jaw of the end effector of the instrument of FIG. 1.

FIG. 4 depicts a perspective view of a second jaw of the end effector of the instrument of FIG. 1.

FIG. 5 depicts an exploded view of the second jaw of FIG. 4.

FIG. 6 depicts another exploded view of the second jaw of FIG. 4.

FIG. 7 depicts a cross-sectional view of the second jaw of FIG. 4, taken along line 7-7 of FIG. 4.

FIG. 8 depicts a cross-sectional view of the second jaw of FIG. 4, taken along line 8-8 of FIG. 4.

FIG. 9 depicts an exploded view of an example of an alternative second jaw that may be incorporated into the end effector of the instrument of FIG. 1.

FIG. 10 depicts an exploded view of another example of an alternative second jaw that may be incorporated into the end effector of the instrument of FIG. 1.

### DETAILED DESCRIPTION

Any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

### I. Overview of Electrosurgical Instrument

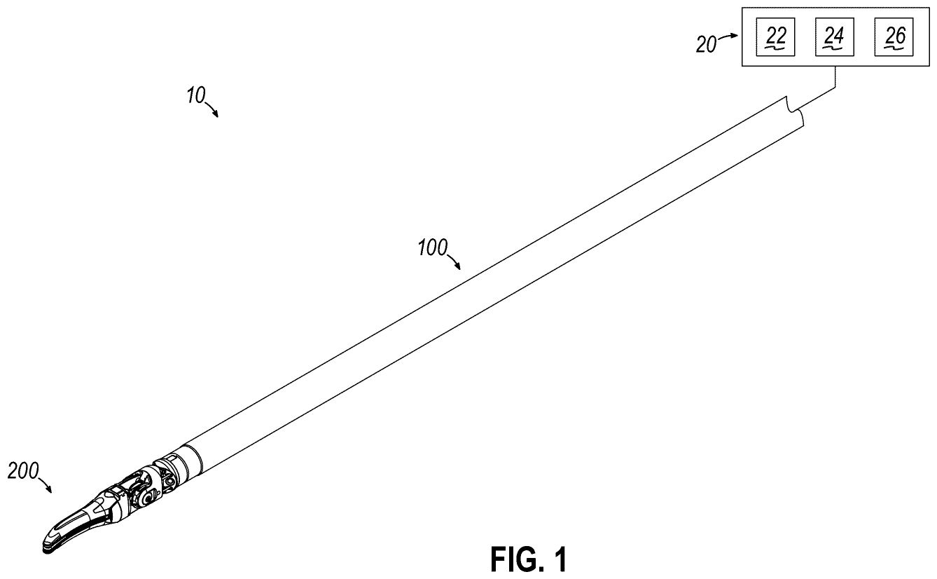

FIG. 1 shows an example of an electrosurgical instrument (10) that may be used in numerous kinds of medical procedures. Instrument (10) of this example includes a body assembly (20), a shaft assembly (100), and an end effector (200). Shaft assembly (100) extends distally relative to body assembly (20). End effector (200) is positioned at a distal end of shaft assembly (100). As will be described in greater detail below, end effector (200) is operable to grasp, cut, and seal or weld tissue (e.g., a blood vessel, etc.). Specifically, end effector (200) is operable to grasp tissue between opposing jaws (220, 240), cut the tissue with a knife (184), and seal or weld the tissue by applying monopolar RF energy to a tissue electrode assembly (244) of jaw (240). While not shown in FIG. 1, instrument (10) may be coupled with a generator that is operable to provide the RF energy to electrode assembly (244). In addition, a ground patch, ground pad, or other electrically grounding feature may be in contact with the patient during use of instrument (10) to provide a ground return for the RF energy applied via tissue electrode assembly (244). In some other versions, jaw (220) also includes an electrode, such that end effector (200) may be operable to apply bipolar RF energy to tissue.

Body assembly (20) may take numerous different forms. In some versions, body assembly (20) includes a handle, such that body assembly (20) is configured to be grasped and manipulated by at least one hand of a human operator. By way of example only, such a handle may include a pistol grip, a scissor grip, or any other suitable configuration. In some other versions, body assembly (20) is configured to be coupled with a robotic arm or other component of a robotic surgical system, such that body assembly (20) is configured to be supported and activated by the robotic surgical system. Alternatively, body assembly (20) may take any other suitable form.

Body assembly (20) of the present example includes a jaw closure assembly (22), a knife drive assembly (24), and an input interface (26). Jaw closure assembly (22) is operable to drive jaws (220, 240) of end effector between an open position and a closed position, with closure motion from jaw closure assembly (22) being communicated to end effector (200) via shaft assembly (100). Any suitable components and arrangements may be used to form jaw closure assembly (22) as will be apparent to those skilled in the art in view of the teachings herein. Knife drive assembly (24) is operable to drive knife (184) between a proximal position and a distal position, with translational motion from knife drive assembly (24) being communicated to end effector (200) via shaft assembly (100). Any suitable components and arrangements may be used to form knife drive assembly (24) as will be apparent to those skilled in the art in view of the teachings herein.

Input interface (26) is operable to interface with a human or robotic operator to activate jaw closure assembly (22), knife drive assembly (24), and/or communication of RF energy to electrode assembly (244). In versions where body assembly (20) is configured to be grasped and manipulated by at least one hand of a human operator, input interface (26) may include one or more triggers, one or more levers, one or more sliders, one or more buttons, and/or any other suitable kind(s) of input features, including combinations thereof. In versions where body assembly (20) is configured to be coupled with a robotic arm or other component of a robotic surgical system, input interface (26) may include one or more gears, one or more drive spindles, one or more shuttles or other translating drive elements, and/or other kind(s) of features that mechanically interface with complementary features of the robotic arm or other component of the robotic surgical system. In addition, or in the alternative, in versions where body assembly (20) is configured to be coupled with a robotic arm or other component of a robotic surgical system, input interface (26) may include one or more pins or sockets, one or more other kinds of electrical contacts, and/or any other kind(s) of features that electrically interface with complementary features of the robotic arm or other component of the robotic surgical system.

### II. Overview of End Effector

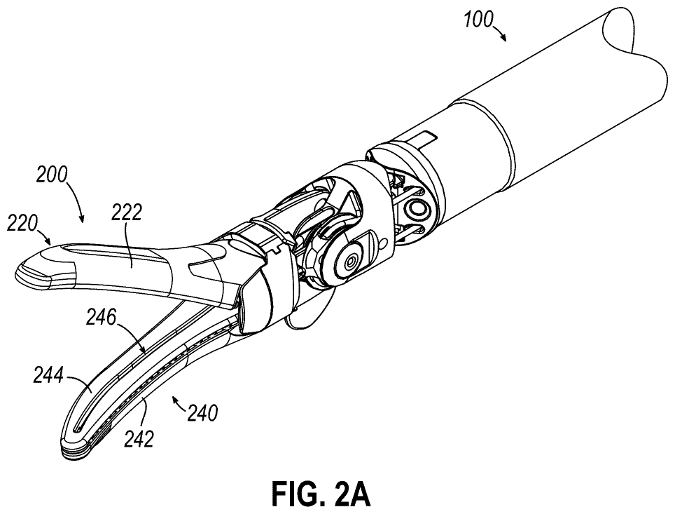

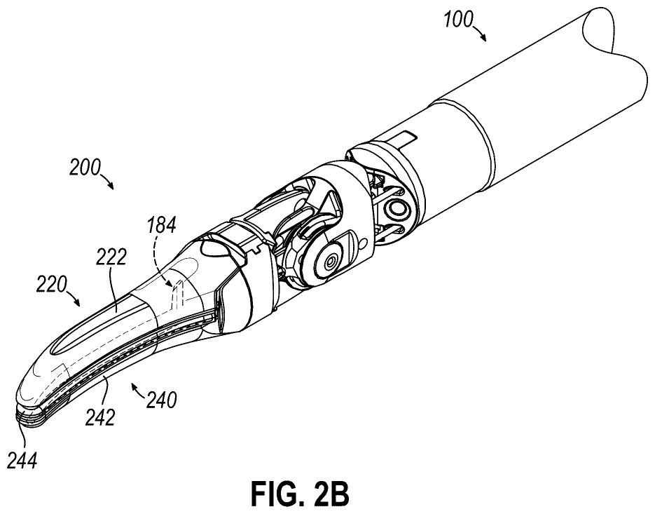

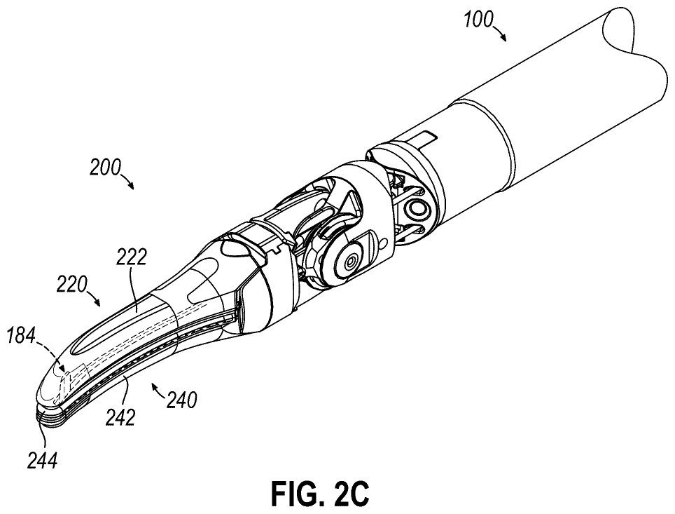

As shown in FIGS. 2A-2C, and as noted above, end effector (200) of the present example includes a first jaw (220) and a second jaw (240) that are operable to transition between an open configuration (FIG. 2A) and a closed configuration (FIG. 2B). In the present example, jaws (220, 240) each pivot toward and away from each other and relative to shaft assembly (100), as jaws (220, 240) transition between the open configuration and the closed configuration. In some other versions, only one of jaws (220, 240) pivot relative to shaft assembly (100) while the other remains stationary to thus transition between the open configuration and the closed configuration. Alternatively, one or both of jaws (220, 240) may move in any other suitable fashion.

As shown in the transition from FIG. 2B to FIG. 2C, knife (184) is operable to translate relative to jaws (220, 240) from a proximal position (FIG. 2B) to a distal position (FIG. 2C). While knife (184) is shown as being in the distal position while jaws (220, 240) are in the open configuration, some versions of operation may provide translation of knife (184) from the proximal position to the distal position only when jaws (220, 240) are in the closed configuration. In other words, in some versions of instrument (10) and methods of use of instrument (10), knife (184) would not be in the distal position when jaws (220, 240) are in the open configuration. The arrangement shown in FIG. 2C should therefore be understood as only being provided for purposes of illustration to show a distal position of knife (184) in relation to jaw (240).

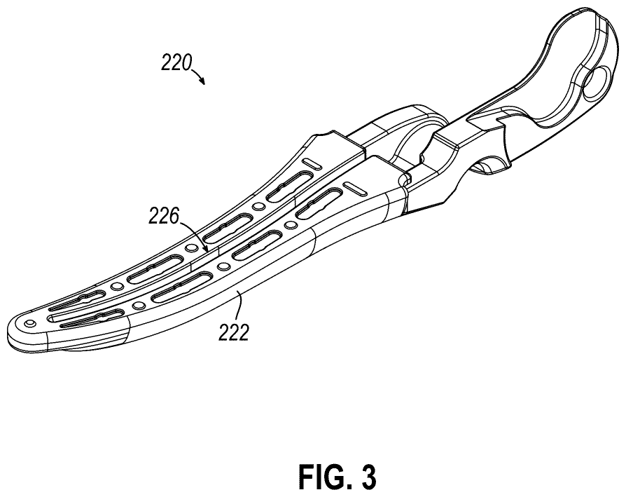

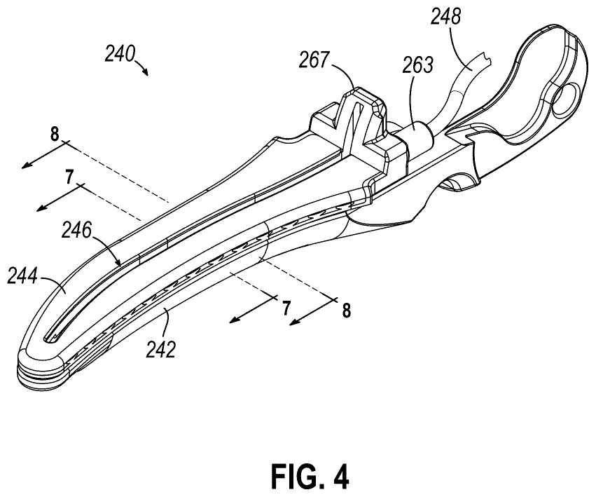

As shown in FIGS. 2A-4 jaw (220) includes a body (222). In the present example, body (222) curves laterally away from a central longitudinal axis along a distal portion of the length of jaw (220). In some other versions, body (222) is substantially straight along the entire length of jaw (220).

Jaw (220) defines a knife slot (226). Knife slot (226) is configured to accommodate longitudinal motion of knife (184) relative to jaw (220) while jaws (220, 240) are in the closed configuration.

As shown in FIGS. 2A-2C and 4-8 jaw (240) includes a body (242) and an electrode assembly (244). Electrode assembly (244) of this example includes an electrode contact (260) and an insulative body (262). Electrode contact (260), also referred to as a conductor, includes an electrically conductive material (e.g., metal, etc.). Insulative body (262) includes an electrically insulative material (e.g., polymer, etc.). Insulative body (262) may be interposed between body (242) and electrode contact (260) to electrically isolate an electrically conductive portion of body (242) relative to electrode contact (260). For instance, insulative body (262) may include insulative plastic material which may be overmolded over a portion of electrode contact (260) and electrode assembly (244) may thereafter be secured to the other side of insulative body (262), such that the plastic material is interposed between electrode contact (260) and the metallic portion of body (242). In the present example, body (242) and electrode assembly (244) curve laterally away from a central longitudinal axis along a distal portion of the length of jaw (240). In some other versions, these features are substantially straight along the entire length of jaw (240).

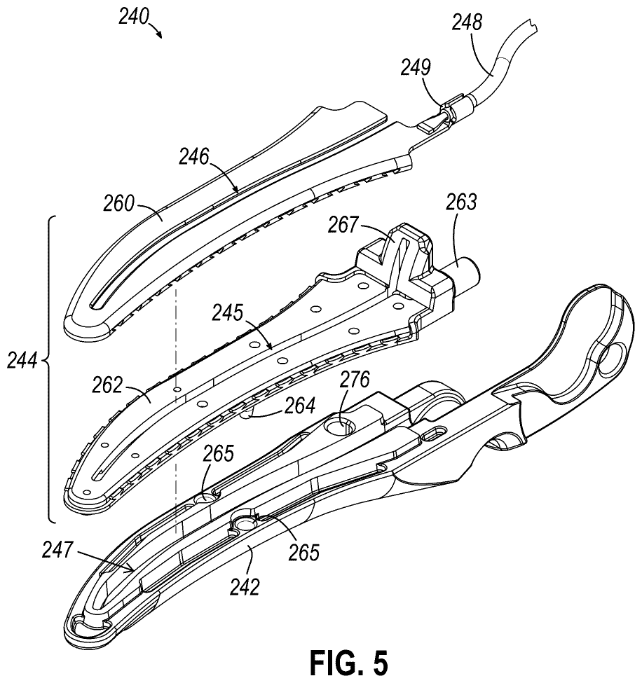

A wire (248) extends proximally from jaw (240). Wire (248) is coupled with electrode assembly (244) via a joint (249), shown as a crimp joint in FIG. 5, and extends along the length of shaft assembly (100) as described in greater detail below. Wire (248) is further configured to couple with a source of RF energy (e.g., an external generator), such that wire (248) is configured to provide RF energy to electrode assembly (244). Insulative body (262) may including a strain relief cuff (263) to secure wire (248) to electrode contact (260) at joint (249). Strain relief cuff (263) surrounds joint (249) to thereby electrically insulate a distal portion of wire (248). In some cases, strain relief cuff (263) may be adhered to an insulation of wire (248) to thereby absorb any stress or strain applied to wire (248) rather than the stress or strain being applied to joint (249). In addition, or in the alternative, strain relief cuff (263) may prevent bending of joint (249) without necessarily being adhered wire (248).

As best seen in FIGS. 5 and 7-8, electrode contact (260) defines a knife slot (246), insulative body (262) defines a knife slot (245), and body (242) defines a knife channel (247). Knife slots (245, 246) align with each other and with knife channel (247) when jaw (240) is fully assembled. Knife slots (245, 246) and knife channel (247) are configured to accommodate longitudinal motion of knife (184) relative to jaw (240) from a proximal position to a distal position. Insulative body (262) of the present example includes a housing (267) at a proximal end to house knife (184) while knife (184) is in the proximal position. A distal surface of housing (267) is distal to a distal cutting edge of knife (184) to thereby cover the distal cutting edge of knife (184) when knife (184) is in the proximal position.

In use, tissue (e.g., a vessel, etc.) may be positioned between jaws (220, 240) while jaws (220, 240) are in an open configuration. Then, jaws (220, 240) may be driven to the closed configuration to clamp the tissue. While jaws (220, 240) remain closed, knife (184) may be driven from the proximal position to the distal position to sever the tissue. In addition, RF energy may be applied to the tissue via electrode assembly (244) to thereby seal the tissue. In some examples of use, RF energy may be applied to the tissue via electrode assembly (244) without knife (184) being advanced distally through the tissue. In either scenario, jaws (220, 240) may be returned to the open configuration to release the tissue when appropriate.

### III. Example of Jaw Assembly Arrangements

As noted above, alignment of components within electrosurgical instruments may be important to promote appropriate function and longevity of the instrument. In the context of end instrument (10), it may be desirable to ensure that knife slots (245, 246) are appropriately aligned with knife channel (247). Such alignment may reduce friction that might otherwise be encountered by knife (184) as knife (184) is advanced distally along jaws (220, 240) and/or may avoid wear that might otherwise be provided on the distal cutting edge of knife (184) when knife (184) is advanced distally along jaws (220, 240). To that end, jaw (240) of the present example may include features that are configured to promote such alignment, as will be described in greater detail below.

As also noted above, some product components that are manufactured through an injection molding process may include an appendage formed by molding material that hardened in a gate of the injection mold. The examples described below provide ways in which such appendages from injection mold gates may be used to provide alignment between knife slots (245, 246) and knife channel (247).

### A. First Example of Alignment Features for Jaw Assembly

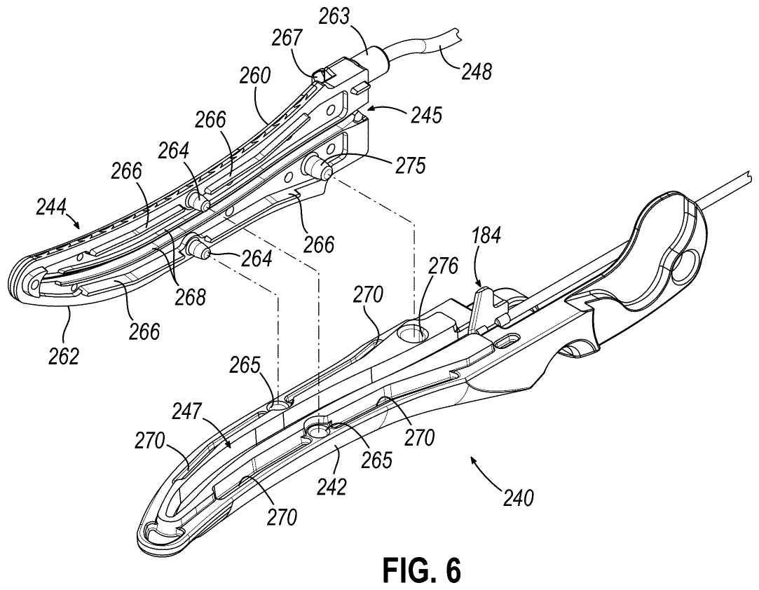

As shown in FIG. 6, to provide alignment between knife slots (245, 246) and knife channel (247), protrusions (264, 275) of electrode assembly (244) may fit into corresponding recesses (265, 276) of body (242). Specifically, insulative body (262) includes intermediate protrusions (264) and proximal protrusion (275). Proximal protrusion (275) is positioned in a proximal region of insulative body (262), while proximal recess (276) is positioned in a proximal region of body (242). Intermediate protrusions (264) are each positioned on opposing sides of knife slot (245) and are longitudinally positioned intermediately between the proximal end of insulative body (262) and the distal end of insulative body (262). Similarly, intermediate recesses (265) are each positioned on opposing sides of knife channel (247) and are longitudinally positioned intermediately between the proximal end of body (242) and the distal end of body (242). By including the three protrusions (264, 275) and three recesses (265, 276) in this arrangement, and by positioning protrusions (264, 275) in corresponding recesses (265, 276) of body (242), shifting of electrode assembly (244) relative to body (242) may be inhibited or prevented.

In some versions, protrusions (264, 275) may be formed in injection molding gates of an injection mold that is used to overmold insulative body (262) against electrode contact (260). In other words, the injection mold cavity may contain electrode contact (260) and an adjacent form for insulative body (262), and the molding material may enter this cavity via gates that are positioned where protrusions (264, 275) will protrude from body (262). The molding material may harden within the cavity (to form insulative body (262)) and within the gates (to form protrusions (264, 275)), thereby forming electrode assembly (244). Thus, rather than being removed like some conventional injection mold gate appendages, protrusions (264, 275) may remain integral with insulative body (262) in the final product.

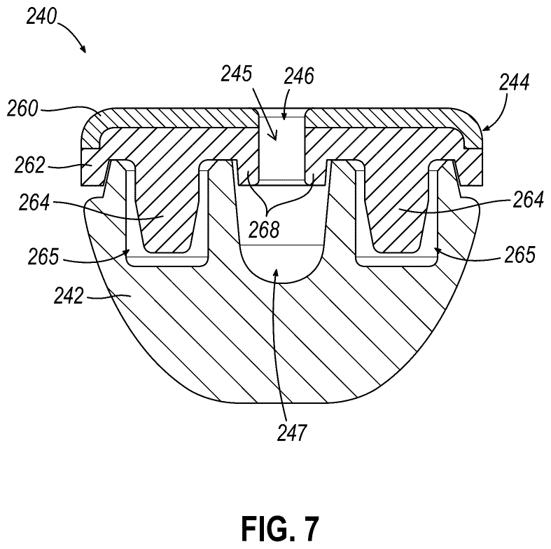

As best seen in FIG. 7, protrusions (264) of the present example have a tapered region near the free end of each protrusion (264). Protrusion (275) may have a similar tapered region. In addition, or in the alternative, the free end of each protrusion (264, 275) may be radiused. Such tapered and/or radiused geometry may promote guidance of protrusions (264, 275) into respective recesses (265, 276) of body (242) during assembly. Protrusions (264, 275) may also be tapered along their whole height to aid in releasing the protrusion (264, 275) from an injection mold. By way of example only, this taper may range from about 1 degrees to about 5 degrees, from the base of the protrusion (264, 275) to the tip of the protrusion (264, 275). Alternatively, any other suitable taper angle may be used.

Protrusions (264, 275) may be press-fit or slip-fit into respective recesses (265, 276) or may be loose fitting. In any scenario, protrusions (264, 275) may be fixedly secured within corresponding recesses (265, 276) via an adhesive, epoxy, or other kind of bonding material; or using any other suitable bonding process. As shown in FIG. 7, a gap is defined between each protrusion (264) and the walls and floor of the corresponding recess (265). A similar gap may be defined between protrusion (275) and the walls and floor of recess (276). Such gaps may receive adhesive, etc., to secure electrode assembly (244) to body (242) as noted above. In some versions, the free end of each protrusion (264, 275) may further define a cavity to receive additional adhesive, etc., to thereby capture the adhesive between the free end of protrusion (264, 275) and the opposing face of the corresponding recess (265, 276) in which protrusion (264, 275) is disposed. In versions where protrusions (264, 275) are press-fit into recesses (265, 276), gaps such as those shown in FIG. 7 may be omitted.

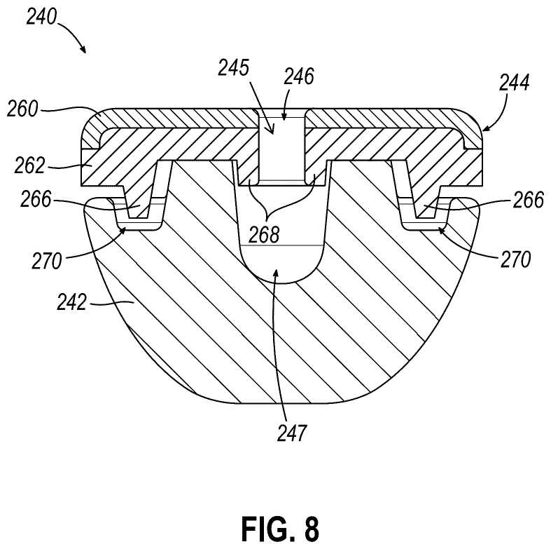

To further aid in alignment between electrode assembly (244) and body (242), insulative body (262) of the present example includes railings (266) along a perimeter; and ribs (268) along knife slot (245) of insulative body (262). As best seen in FIG. 8, railings (266) are received in and adhered to respective troughs (270) of body (242). As shown in FIG. 8, a gap is defined between each railing (266) and the walls and floor of the corresponding trough (270). Such gaps may receive adhesive, etc., to secure electrode assembly (244) to body (242) as noted above. In some other versions, such gaps may be omitted.

As also shown in FIG. 8 (and in FIG. 7), ribs (268) are positioned along knife slot (245) and are received in knife channel (247) of body (242) to thereby act as a guide for knife (184) and further promote alignment between electrode assembly (244) and body (242). In the present example, ribs (268) extend along a substantial length of knife channel (247), such that ribs (268) extend along the entire range of travel of knife (184) within knife channel (247).

### B. Second Example of Alignment Features for Jaw Assembly

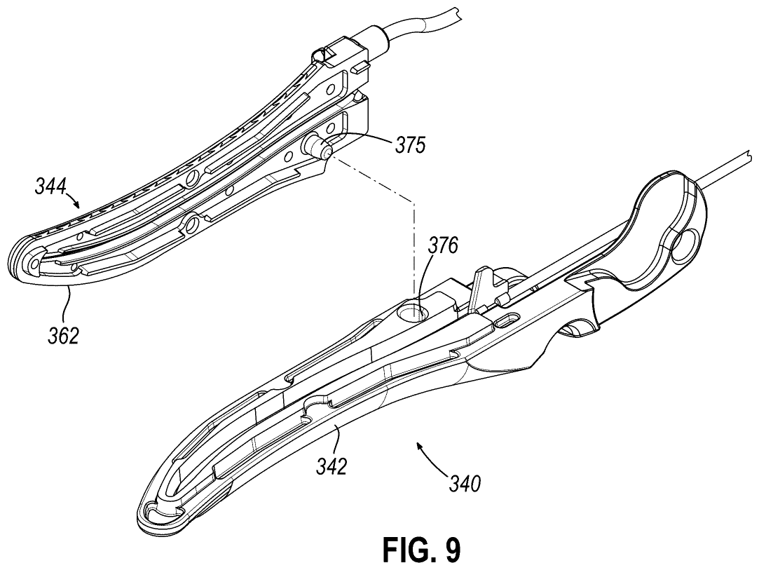

FIG. 9 shows another example of a second jaw (340) that may be incorporated into end effector (200) in place of second jaw (240). Second jaw (340) may be constructed and operable substantially the same as second jaw (240) but with the following differences. Insulative body (362) of electrode (344) may include one protrusion (375) positioned on a proximal portion of insulative body (362). Insulative body (362) may lack any protrusions that are equivalent to intermediate protrusions (264) of insulative body (262); or may include a recess in place of intermediate protrusions (264). Body (342) may also lack respective intermediate recesses that are equivalent to intermediate recesses (265), but may also include proximal recess (376), as shown, for receiving protrusion (375).

### C. Third Example of Alignment Features for Jaw Assembly

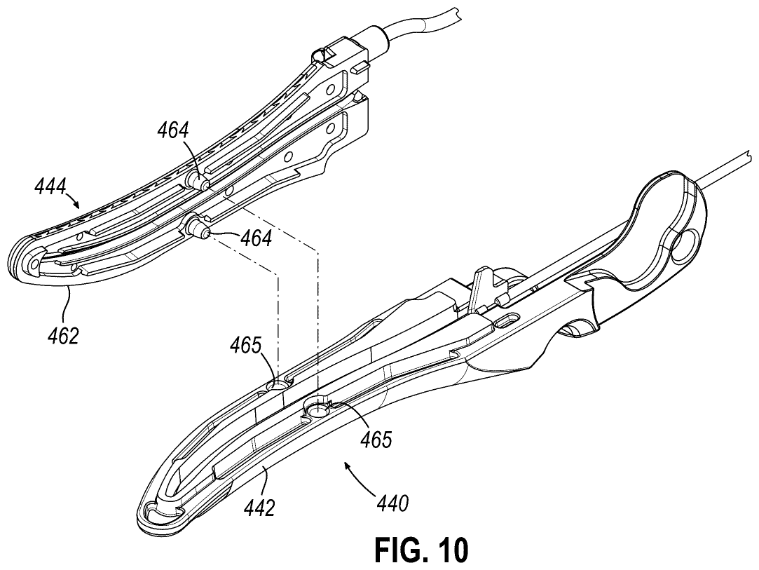

FIG. 10 shows another second jaw (440) that may be incorporated into effector (200) in place of second jaw (240). Second jaw (440) may be configured and operable substantially the same as second jaws (240, 340) but with the following differences. Insulative body (462) of electrode (444) may include two intermediate protrusions (464) positioned on a mid-region of insulative body (462). Insulative body (462) may lack any protrusion that is equivalent to proximal protrusion (275, 375) of insulative body (262, 362); or may include a recess in place of proximal protrusion (275, 375). Body (442) may also lack a respective proximal recess that is equivalent to proximal recess (276, 376), but may also include intermediate recesses (465), as shown, for receiving protrusions (464).

### III. Examples of Combinations

The following examples relate to various non-exhaustive ways in which the teachings herein may be combined or applied. The following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An instrument comprising: (a) a body assembly; (b) a shaft extending from the body assembly; and (c) an end effector including: (i) a first jaw, and (ii) a second jaw, the end effector being configured to grasp tissue between the first jaw and the second jaw, the shaft being positioned between the body assembly and the end effector, the second jaw including: (A) a second jaw body, the second jaw body including at least one recess, and (B) an electrode assembly, the electrode assembly including at least one protrusion extending toward the second jaw body, and the at least one protrusion being sized to fit inside a respective recess of the at least one recess and configured to align the electrode assembly to the second jaw body.

### Example 2

The instrument of Example 1, the electrode assembly including an insulative body and a conductor, the conductor being electrically conductive, the insulative body being electrically insulative, the insulative body being affixed to the second jaw body to thereby separate the conductor from the second jaw body.

### Example 3

The instrument of Example 2, the insulative body being coupled to the conductor, the at least one protrusion extending integrally from the insulative body.

### Example 4

The instrument of any of Examples 1 through 3, the electrode assembly including a knife slot, the at least one protrusion including two protrusions, the two protrusions being positioned on opposing lateral sides of the knife slot.

### Example 5

The instrument of Example 4, the at least one protrusion including a third protrusion positioned proximal to the two protrusions such that the third protrusion is nearer the shaft.

### Example 6

The instrument of any of Examples 1 through 5, a protrusion of the at least one protrusion being configured to press-fit into a recess of the at least one recess.

### Example 7

The instrument of any of Examples 1 through 6, the second jaw further including an adhesive interposed between the electrode assembly and the second jaw body, the adhesive securing the electrode assembly to the second jaw body.

### Example 8

The instrument of any of Examples 1 through 7, further comprising an electrical conduit extending along the shaft, the electrical conduit being configured to supply electricity to the electrode assembly.

### Example 9

The instrument of Example 8, the electrode assembly further including an insulative body and a conductor, the conductor being electrically conductive and in electrical communication with the electrical conduit, the insulative body being electrically insulated, the insulative body being positioned around a distal portion of the electrical conduit to thereby electrically insulate the distal portion of the electrical conduit.

### Example 10

The instrument of any of Examples 1 through 9, the electrode assembly including a knife slot extending along a portion of a length of the electrode assembly, the instrument further comprising a knife configured to translate along the knife slot.

### Example 11

The instrument of Example 10, the second jaw body including a knife channel, the electrode assembly including a pair of ribs positioned along opposing sides of the knife slot, the pair of ribs being sized to fit inside the knife channel and configured to guide the knife during translation of the knife along the knife slot.

### Example 12

The instrument of Example 11, the pair of ribs being a portion of the insulative body.

### Example 13

The instrument of any of Examples 10 through 12, the knife being configured to translate between a proximal position to a distal position, the electrode assembly being configured to house the knife while in the proximal position.

### Example 14

The instrument of Example 13, the electrode assembly including an insulative body affixed to a conductor, the conductor being electrically conductive, the insulative body being electrically insulated, the insulative body being affixed to the second jaw body to thereby separate the conductor from the second jaw body, at least a portion of the knife being positionable in the insulative body.

### Example 15

The instrument of any of Examples 13 through 14, further comprising an electrical conduit configured to provide electricity to the electrode assembly, the insulative body including a strain relief cuff surrounding the electrical conduit, the strain relief cuff being configured to relieve stress or strain on the electrical conduit.

### Example 16

An instrument comprising: (a) a body assembly; (b) a shaft extending from the body assembly; (c) a knife; and (d) an end effector including: (i) a first jaw, and (ii) a second jaw, the end effector being configured to grasp tissue between the first jaw and the second jaw, the shaft being positioned between the body assembly and the end effector, the second jaw defining a knife slot for the knife to translate within, the second jaw including: (A) a second jaw body, the second jaw body including a knife channel sized to fit the pair of ribs, the knife channel extending along the knife slot, and (B) an electrode assembly, the electrode assembly including a pair of ribs extending toward the second jaw body and along the knife slot.

### Example 17

A method of producing a jaw of a surgical ablation instrument, the method including: (a) overmolding an insulative body onto a conductor to form an electrode assembly, the conductor being electrically conductive, the insulative body being electrically insulating, the act of overmolding including introducing a molding material into a mold cavity via one or more mold gates, at least a portion of the conductor being positioned in the mold cavity, the overmolding process resulting in one or more protrusions extending from the insulative body, the one or more protrusions being formed by molding material in the one or more mold gates; (b) providing a second jaw body, the second jaw body including one or more recesses; (c) inserting the one or more protrusions of the electrode assembly into the one or more recesses of the second jaw body; and (d) securing the electrode assembly to the second jaw body with the one or more protrusions of the electrode assembly disposed on the one or more recesses of the second jaw body.

### Example 18

The method of Example 17, the act of securing the electrode assembly to the second jaw body comprising adhering the one or more protrusions of the electrode assembly in the one or more recesses of the second jaw body.

### Example 19

The method of any of Examples 17 through 18, the insulative body including a railing, the second jaw body including a trough, the method for including positioning the railing inside the trough.

### Example 20

The method of any of Examples 17 through 19, the insulative body including a rib defining an electrode knife slot, the second jaw body including a jaw knife channel, the method including positioning the rib inside the jaw knife channel to thereby align the electrode knife slot with the jaw knife channel.

### IV. Miscellaneous

Any of the versions of the instruments described herein may include various other features in addition to or in lieu of those described above. While the examples herein are described mainly in the context of electrosurgical instruments, various teachings herein may be readily applied to a variety of other types of devices. By way of example only, the various teachings herein may be readily applied to other types of electrosurgical instruments, tissue graspers, tissue retrieval pouch deploying instruments, surgical staplers, surgical clip appliers, ultrasonic surgical instruments, etc. The teachings herein may be readily applied to any of the instruments described in any of the references cited herein. Any of the devices herein may also include one or more of the various features disclosed in any of the various references that are incorporated by reference herein. The teachings herein may thus be readily combined with the teachings of any of the references cited herein in numerous ways. Other types of instruments into which the teachings herein may be incorporated will be apparent to those skilled in the art in view of the teachings herein.

It should be appreciated that any patent, publication, or other disclosure material, in whole or in part, that is said to be incorporated by reference herein is incorporated herein only to the extent that the incorporated material does not conflict with existing definitions or other disclosure material set forth in this disclosure. The disclosure as explicitly set forth herein supersedes any conflicting material incorporated herein by reference. Any material, or portion thereof, that is said to be incorporated by reference herein, but which conflicts with existing definitions or other disclosure material set forth herein will only be incorporated to the extent that no conflict arises between that incorporated material and the existing disclosure.

The terms “proximal” and “distal” are defined herein relative to a surgeon, robotic arm, or other operator or structure grasping a surgical instrument having a distal surgical end effector. The term “proximal” refers the position of an element closer to the surgeon, robotic arm, or other operator or structure; and the term “distal” refers to the position of an element closer to the surgical end effector of the surgical instrument and further away from the surgeon or other operator or structure.

As used herein, an element or step recited in the singular and proceeded with the word “a” or “an” should be understood as not excluding plural of said elements or steps, unless such exclusion is explicitly stated. Furthermore, references to “one example” or “an example” are not intended to be interpreted as excluding the existence of additional examples that also incorporate the recited features. The use of “including,” “comprising,” or “having” and variations thereof herein is meant to encompass the items listed thereafter, and equivalents thereof, as well as additional items.

As used herein in the specification and claims, including as used in the examples and unless otherwise expressly specified, the terms “about” or “approximately” for any numerical values or ranges indicate a suitable dimensional tolerance, or other form of reasonable expected range, that allows the part or collection of components to function for its intended purpose as described herein. More specifically, “about” or “approximately” may refer to the range of values that are within ±10% of the recited value (e.g., “about 100” may refer to the range of values from 90 to 110, including 90, 110, 100, and all other values within the range of 90 and 110). Any numerical values given herein should also be understood to include about or approximately that value unless the context indicates otherwise. For example, if the value “10” is disclosed, then “about 10” is also disclosed. Any numerical range recited herein is intended to include all sub-ranges subsumed therein. The terms “approximately” and “about” are thus utilized herein to represent the degree by which a quantitative representation may vary from a stated reference without resulting in a change in the basic function of the subject matter at issue.

The term “substantially” is also utilized herein to represent the degree by which a quantitative representation may vary from a stated reference without resulting in a change in the basic function of the subject matter at issue. The term “substantially” shall therefore be understood to include a range of conditions or results that provide a functional equivalent to an explicitly stated condition or result. For instance, if a task is “substantially complete,” the result of the task having been substantially completed is functionally equivalent to the result that would have been achieved if the task had been perfectly completed. As another non-limiting example, a component that is “substantially straight” or “substantially flat,” an apparatus including a component that is “substantially straight” or “substantially flat” may provide a result or effect that is functionally equivalent to a result or effect that would be achieved by the same apparatus including the same component in a perfectly straight or perfectly flat configuration. The range implied by the term “substantially” should also be read to include the perfect result that is within that range. Thus, the term “substantially complete” shall be read as including “perfectly complete” while also including a range of completeness that is functionally equivalent to perfectly complete. As another example, terms such as “substantially straight” and “substantially flat” shall be read as including “perfectly straight” and “perfectly flat,” respectively; while also including a range of straightness or flatness that is functionally equivalent to perfectly straight or flat, respectively. As with the terms “approximately” and “about,” the term “substantially” may indicate a suitable dimensional tolerance, or other form of reasonable expected range, that allows a part or collection of components to function for its intended purpose as described herein.

The limitations of the following claims are not written in means-plus-function format and are not intended to be interpreted based on 35 U.S. C. § 112(f), unless such claim limitations expressly use the phrase “means for” followed by a statement of function void of further structure.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

What is claimed is:

1. An instrument comprising:
(a) a body assembly;
(b) a shaft extending from the body assembly; and
(c) an end effector including:
(i) a first jaw, and
(ii) a second jaw, the end effector being configured to grasp tissue between the first jaw and the second jaw, the shaft being positioned between the body assembly and the end effector, the second jaw including:
(A) a second jaw body, the second jaw body including at least one recess, and
(B) an electrode assembly, the electrode assembly including at least one protrusion extending toward the second jaw body, and the at least one protrusion being sized to fit inside a respective recess of the at least one recess and configured to align the electrode assembly to the second jaw body.

2. The instrument of claim 1, the electrode assembly including an insulative body and a conductor, the conductor being electrically conductive, the insulative body being electrically insulative, the insulative body being affixed to the second jaw body to thereby separate the conductor from the second jaw body.

3. The instrument of claim 2, the insulative body being coupled to the conductor, the at least one protrusion extending integrally from the insulative body.

4. The instrument of claim 1, the electrode assembly including a knife slot, the at least one protrusion including two protrusions, the two protrusions being positioned on opposing lateral sides of the knife slot.

5. The instrument of claim 4, the at least one protrusion including a third protrusion positioned proximal to the two protrusions such that the third protrusion is nearer the shaft.

6. The instrument of claim 1, a protrusion of the at least one protrusion being configured to press-fit into a recess of the at least one recess.

7. The instrument of claim 1, the second jaw further including an adhesive interposed between the electrode assembly and the second jaw body, the adhesive securing the electrode assembly to the second jaw body.

8. The instrument of claim 1, further comprising an electrical conduit extending along the shaft, the electrical conduit being configured to supply electricity to the electrode assembly.

9. The instrument of claim 8, the electrode assembly further including an insulative body and a conductor, the conductor being electrically conductive and in electrical communication with the electrical conduit, the insulative body being electrically insulated, the insulative body being positioned around a distal portion of the electrical conduit to thereby electrically insulate the distal portion of the electrical conduit.

10. The instrument of claim 1, the electrode assembly including a knife slot extending along a portion of a length of the electrode assembly, the instrument further comprising a knife configured to translate along the knife slot.

11. The instrument of claim 10, the second jaw body including a knife channel, the electrode assembly including a pair of ribs positioned along opposing sides of the knife slot, the pair of ribs being sized to fit inside the knife channel and configured to guide the knife during translation of the knife along the knife slot.

12. The instrument of claim 11, the pair of ribs being a portion of the insulative body.

13. The instrument of claim 10, the knife being configured to translate between a proximal position to a distal position, the electrode assembly being configured to house the knife while in the proximal position.

14. The instrument of claim 13, the electrode assembly including an insulative body affixed to a conductor, the conductor being electrically conductive, the insulative body being electrically insulated, the insulative body being affixed to the second jaw body to thereby separate the conductor from the second jaw body, at least a portion of the knife being positionable in the insulative body.

15. The instrument of claim 13, further comprising an electrical conduit configured to provide electricity to the electrode assembly, the insulative body including a strain relief cuff surrounding the electrical conduit, the strain relief cuff being configured to relieve stress or strain on the electrical conduit.

16. An instrument comprising:
(a) a body assembly;
(b) a shaft extending from the body assembly;
(c) a knife; and
(d) an end effector including:
(i) a first jaw, and
(ii) a second jaw, the end effector being configured to grasp tissue between the first jaw and the second jaw, the shaft being positioned between the body assembly and the end effector, the second jaw defining a knife slot for the knife to translate within, the second jaw including:
(A) a second jaw body, the second jaw body including a knife channel sized to fit the pair of ribs, the knife channel extending along the knife slot, and
(B) an electrode assembly, the electrode assembly including a pair of ribs extending toward the second jaw body and along the knife slot.

17. A method of producing a jaw of a surgical ablation instrument, the method including:
(a) overmolding an insulative body onto a conductor to form an electrode assembly, the conductor being electrically conductive, the insulative body being electrically insulating, the act of overmolding including introducing a molding material into a mold cavity via one or more mold gates, at least a portion of the conductor being positioned in the mold cavity, the overmolding process resulting in one or more protrusions extending from the insulative body, the one or more protrusions being formed by molding material in the one or more mold gates;
(b) providing a second jaw body, the second jaw body including one or more recesses;
(c) inserting the one or more protrusions of the electrode assembly into the one or more recesses of the second jaw body; and
(d) securing the electrode assembly to the second jaw body with the one or more protrusions of the electrode assembly disposed on the one or more recesses of the second jaw body.

18. The method of claim 17, the act of securing the electrode assembly to the second jaw body comprising adhering the one or more protrusions of the electrode assembly in the one or more recesses of the second jaw body.

19. The method of claim 17, the insulative body including a railing, the second jaw body including a trough, the method for including positioning the railing inside the trough.

20. The method of claim 17, the insulative body including a rib defining an electrode knife slot, the second jaw body including a jaw knife channel, the method including positioning the rib inside the jaw knife channel to thereby align the electrode knife slot with the jaw knife channel.
